(19) United States
(12) Patent Application Publication
(10) Pub. No.: US 2021/0002596 A1
(43) Pub. Date: Jan. 7, 2021

(54) Mold and Method for Preparing a Hollow 3D Cell Tissue Structure

(71) Applicant: Universiteit Twente, Enschede (NL)

(72) Inventors: Marcelo Catarino Ribeiro, Amersfoort (NL); Petrus Christianus Johannes Josephus Passier, Driebergen-Rijsenburg (NL)

(21) Appl. No.: 16/977,884

(22) PCT Filed: Mar. 7, 2019
(86) PCT No.: PCT/NL2019/050141

§ 371 Date: Sep. 3, 2020

(30) Foreign Application Priority Data
Mar. 7, 2018 (EP) 18160489.3

(51) Int. Cl. C12M 3/00; C12N 5/077; C12N 5/071; A61L 27/38; C12N 5/00

(52) U.S. Cl. CPC C12M 21/08; C12N 5/0657; C12N 5/0697; C12N 2513/00; A61L 27/3834; C12N 5/0062; A61L 2430/20; A61L 27/3826

(57) ABSTRACT

The present invention relates to a mold for preparing a hollow 3D cell tissue structure such as an organoid, and uses thereof. Methods for preparing a hollow 3D cell tissue structure such as an organoid, in particular a human heart mimic, are also provided.

The invention relates to a mold for preparing a hollow 3D cell tissue structure such as an organoid, in particular a human heart mimic, comprising an external body having an inner surface delimiting a cavity in the external body, and an internal body having an outer surface, wherein the internal body is positioned in the cavity of the external body, and wherein the inner surface of the external body and the outer surface of the internal body define a culture chamber there between for receiving cells therein. The invention moreover relates to a method for preparing a hollow 3D cell tissue structure such as an organoid, in particular a human heart mimic.

The process of drug discovery and development is often hampered by a low specificity and efficacy of newly developed drugs in combination with unexpected detrimental side effects, leading to a high attrition rate. Consequently, the process of drug discovery and development is usually a very costly and long process. Despite increased worldwide expenditure for development the actual number of new molecular entities reaching the market is declining. For example cardiovascular diseases (both hereditary and acquired disease, including channelopathies, cardiomyopathies, myocardial infarction, among others) are the major cause of morbidity and mortality worldwide, and yet there is still no fully effective treatment available. The occurrence of drug-induced cardiotoxicity is responsible for 30% of drug candidate failures in clinical safety trials, preventing new medicines from reaching patients and causing investment losses of billions of euros.

A major limitation in better understanding the underlying mechanisms of disease as well as avoiding drug-induced cardiotoxicity is the lack of predictable 3D cell tissue models. This limitation arises from the fact that preclinical research cannot be performed in humans due to ethical issues and the absence of age and genetically matched controls. Instead using animal models has as a drawback that these non-human models do not faithfully recapitulate human disease, which severely limits the predictability and effectiveness of these models.

The use of human stem cells and in particular the ground-breaking discovery of the generation of human induced pluripotent stem cells (hiPSC) opened the door to personalized medicine, i.e. using patient-derived cells for human in vitro disease modelling and production of human specialized cell types for regenerative medicine. In past years new procedures have thus been developed to generate 3D tissue structures based on such cells. A known method to make 3D cell tissues is based on casting cells, e.g. hPSC-cardiomyocytes, surrounded by extracellular matrix (ECM) such as Matrigel, collagen I and fibrin, in molds having an internal body positioned in a cavity of an external body, wherein an inner surface of the external body and an outer surface of the internal body define a culture chamber there between for culturing the cell tissue therein. A drawback of the available molds is that a particular 3D structure of the tissue therein is determined by the culture chamber provided between the internal body and external body of the mold. As removal of the mold bodies after forming of the 3D cell tissue structure in the culture chamber may be difficult, particularly without disturbing the formed 3D tissue, when the bodies have complex forms, the available molds are limited to provide 3D cell tissue structures with rather simple and straightforward shapes. For example, shapes featuring angles sharper than 90°, oval shapes or different shape combinations, such as mimicking one ventricle of a heart or a combination of one atrium and one ventricle, are extremely difficult or even impossible to make with the current technology.

The invention is thus directed to means and methods for generating cellular based structures (biological tissues) with a high degree of structural complexity. More specifically, the invention enables the making of cellular based structures with hollow features.

It is a particular object of the present invention to provide an improved mold for preparing a hollow 3D cell tissue structure. Another particular object of the present invention is to provide an improved mold for preparing an organoid, particularly a human organoid. A further particular object of the present invention is to provide an improved mold for preparing a human heart mimic.

To meet these and other objects and aspects of the invention the invention provides a mold as defined in one or more of the appended claims. Also provided are a method, a use and a bioreactor as defined in one or more of the appended claims.

Particularly, according to the invention there is provided a mold for preparing a hollow 3D cell tissue structure such as an organoid, in particular a human heart mimic, the mold comprising an external body having an inner surface delimiting a cavity in the external body, and an internal body having an outer surface, wherein the internal body is positioned in the cavity of the external body, and wherein the inner surface of the external body and the outer surface of the internal body define a culture chamber there between for receiving cells therein, which mold is characterized in that the internal body is made in a predefined shape of a fugitive material to enable degradation of at least part of the internal body without affecting an integrity of a formed cell structure and/or cell tissue. The mold according to the invention combines the degradability or solubility of biocompatible fugitive materials with cells in order to allow forming of three-dimensional biological tissues with complex shapes and features. A mold according to the present invention is therewith particularly suitable for preparing an organoid. Since a mold according to the invention comprises a degradable internal body made of fugitive material, it is particularly suitable for preparing an organoid with a cavity, or a hollow organoid. Non-limiting examples of such organoids are cardiac organoids, renal organoids, lung organoids, gut organoids, bladder organoids and eye organoids. In some preferred embodiments, a human organoid is prepared with a mold according to the invention. As used herein, a human organoid is defined as an organoid comprising cells that are derived from human cells, for instance from human stem cells and/or from human progenitor cells and/or from more differentiated human cells such as for instance from growing (dividing) human tissue cells. In particular, the mold according to the invention allows preparing of a human heart mimic with hollow structures representing the heart's cavities allowing cardiomyocytes in the cell tissue to contract and enable the heart's pumping function. The same concept applies for example to the alveolus in the lung or the renal medulla (pyramid) in the kidneys or to the hollow structure of the gut, bladder or the eye.

According to the invention, a 3D cell tissue structure, particularly an organoid, is prepared by a method comprising:
preparing an external body having an inner surface delimiting a cavity in the external body;
preparing an internal body of fugitive material in a predefined shape and having an outer surface;
placing the internal body inside the cavity of the external body, wherein the inner surface and outer surface define a culture chamber there between;
providing cells, particularly stem cells, progenitor cells, cardiomyocytes and/or other cardiac cells in the culture chamber;
subjecting the cells to incubating conditions for a period of time to promote the formation of a supported cell structure, and/or to incubating conditions to promote cell tissue formation, in the culture chamber around the outer surface of the internal body;
subjecting the internal body of fugitive material to a condition whereto the fugitive material reacts thereby degrading the internal body for removal from the cavity without significantly affecting an integrity of the formed supported cell structure or cell tissue in the culture chamber and maintaining a functioning of the supported cell structure or the formed cell tissue structure.

As used herein, the term “supported cell structure” refers to a structure wherein the mobility of the cells is reduced as compared to the cell mobility in an aqueous solution. Preferably, the mobility of the cells is limited to such extent that they are fixed, meaning that the cells remain within a certain area around a given position. This is preferably accomplished by encapsulation of the cells into a hydrogel. Hence, a preferred supported cell structure is a hydrogel with encapsulated cells.

As used herein, the term “tissue formation” refers to a process wherein cells connect to each other. Typically, tissue formation involves the proliferation of cells and/or attachment of cells to each other, thereby growing a cell tissue structure, preferably an organoid.

Incubating conditions to promote the formation of a supported cell structure for instance comprise conditions that allow for an increase of viscosity of a culture medium, liquid, gelatinous composition or cell suspension, and/or conditions that allow for the formation of a hydrogel.

Incubating conditions to promote cell tissue formation for instance comprise a temperature of about 37 degrees Celsius and/or an environment comprising 5% CO₂ and 21% O₂.

As used herein, the term “without significantly affecting an integrity of the formed supported cell structure or cell tissue” means that the structure and/or the function of the formed supported cell structure or cell tissue is not significantly altered as a result of degradation of the internal body. In preferred embodiments, the degradation of the internal body does not adversely affect the integrity of the supported cell structure or cell tissue in the culture chamber, meaning that degradation of the internal body does not result in a measurable loss of cells or a measurable decrease of the function of the supported cell structure or of the cell tissue, as compared to the amount of cells and the function of the supported cell structure or the cell tissue before degradation of the internal body.

Both the mold and method for preparing a hollow 3D cell tissue structure according to the invention are based on the internal body being degradable after formation of the supported cell structure and/or the cell tissue in the culture chamber so that the internal body can be released from the cell structure and/or cell tissue independent of a shape of the supported cell structure or cell tissue structure and without significantly disturbing or affecting the supported cell structure or cell tissue. To this end the internal body is made at least in part, and preferably completely, from a fugitive material. As used herein a fugitive material comprises any material that allows for separation of the material from other materials by a change or transition of the material into a different form or state when a particular condition is applied thereto which particular condition does not significantly affect or change the supported cell structure or cell tissue. For example, the fugitive material may be a material that transitions from a solid state, in which it may form the internal body of the mold according to the invention in the predefined shape, into a fluid state, in which it may be flown out, thereby forming a cavity inside the supported cell structure or cell tissue structure. The particular condition for changing or transitioning the fugitive material may for example be exposure to an enzyme, a reactive chemical compound, a temperature, a pressure, a sound, and/or to light at a certain wavelength. Such conditions may lead to the change or transition of the fugitive material if a certain threshold value of the condition for the fugitive material is exceeded. The fugitive material may particularly be adapted to have a threshold value for one of the conditions that allows the fugitive material to change or transition into the state in which the internal body of the mold is degraded, without significantly disturbing or affecting the supported cell structure or cell tissue at such threshold value, preferably without adversely affecting the function of the formed cell structure or cell tissue.

In a preferred embodiment the mold according to the invention is thus characterized in that the fugitive material is adapted to degrade above or below a temperature threshold value and/or in the presence of an enzyme and/or in the presence of a reactive chemical compound and/or by sonication and/or in the presence of light with a wavelength above or below a threshold value.

Of the possible conditions which may change or transition the fugitive material such that the internal body is degraded, applying a temperature change in many situations may easily be realized. Thus, in a further preferred embodiment the mold according to the invention is characterized in that the fugitive material is adapted to degrade at a temperature between 10-50 degrees Celsius, more particularly between 25-40 degrees Celsius.

In a particular embodiment the mold according to the invention is characterized in that the fugitive material is substantially solid below 10 degrees Celsius and is adapted to dissolve and/or liquefy above 10 degrees Celsius, in particular to dissolve and/or liquefy at a temperature between 10-42 degrees Celsius. For example an internal body made of gelatin may be adapted to be solid below 10 degrees Celsius, forming the internal body in the predefined shape, and to liquefy when exposing the internal body to an increased temperature between 10-42 degrees Celsius, so that the resulting liquid may be flown out of the 3D cell structure or cell tissue structure when a 3D cell structure or cell tissue structure is formed in the mold. A temperature between 10-42 degrees Celsius will for most cell structures and cell tissues not be a disturbing or affecting condition. Particularly viability of the cells will not, or at least not relevantly, be reduced when exposing the cells to such a temperature.

In a further particular embodiment the mold according to the invention is characterized in that the fugitive material is substantially solid above 15 degrees Celsius and is adapted to dissolve and/or liquefy below 15 degrees Celsius. For example an internal body comprising Pluronic® F-127 may be adapted to be solid, particularly gelatinous, above 15 degrees Celsius, forming the internal body in the predefined shape, and to dissolve when exposing the internal body to a temperature below 15 degrees Celsius, for instance using a dissolving fluid at a temperature below 15 degrees Celsius, so that the resulting solution may be flown out of the 3D cell structure or cell tissue structure when a 3D cell structure or cell tissue structure is formed in the mold.

Some particular embodiments thus provide a mold for preparing a hollow 3D cell tissue structure such as an organoid, in particular a human heart mimic, comprising: an external body having an inner surface delimiting a cavity in the external body; an internal body having an outer surface, wherein the internal body is positioned in the cavity of the external body, wherein the inner surface of the external body and the outer surface of the internal body define a culture chamber there between for receiving cells therein, characterized in that the internal body is made in a predefined shape of a degradable material to enable degradation of at least part of the internal body without significantly affecting an integrity of a formed cell structure and/or cell tissue. The degradable material is preferably degradable above or below a temperature threshold value and/or in the presence of an enzyme and/or in the presence of a reactive chemical compound and/or by sonication and/or in the presence of light with a wavelength above or below a threshold value. In some embodiments, the degradable material is degradable at a temperature between 10-50 degrees Celsius, preferably between 25-40 degrees Celsius. In some embodiments, the degradable material is substantially solid below 10 degrees Celsius and dissolves and/or liquefies above 10 degrees Celsius, preferably dissolves and/or liquefies at a temperature between 10-42 degrees Celsius. In some embodiments, the degradable material is substantially solid above 15 degrees Celsius and dissolves and/or liquefies below 15 degrees Celsius.

A further preferred embodiment of the mold according to the invention is characterized in that the fugitive material comprises gelatin, particularly at least 10% gelatin, and/or Pluronic® F-127 and/or poly(vinyl alcohol) (PVA). These fugitive materials are biocompatible with cells and/or cell tissue, and moreover allow forming of parts of the mold in a predefined, or desired, shape with conventional, well-known means, such as simple cutting tools.

A further particular embodiment of the mold according to the invention is characterized in that the internal body and the external body are made of a fugitive material. Thus in addition to the internal body also the external body of the mold may be made of fugitive material in order to allow a degradation thereof to completely release a 3D cell structure or cell tissue structure from the mold when formed therein.

In a further preferred embodiment the mold according to the invention is characterized in that the internal body and the external body are formed from a different fugitive material. Thus, as the internal body and external body of the mold are not made of the same fugitive material, it may be prevented that the internal body and external body degrade simultaneously when exposing the fugitive material to the changing or transitioning condition. Instead, the fugitive materials may have different threshold values for changing or transitioning at the particular condition, or may require different conditions altogether in order to be able to control a degradation of the internal body and external body independently. In an alternative embodiment the mold according to the invention is characterized in that the internal body and the external body are formed from the same fugitive material, thus allowing a one step, or simultaneous, degradation of the internal body and external body. The internal body and the external body formed from the same fugitive material may be degraded independently from each other by applying a degradation condition more locally within the mold, either exposing the internal body or the external body to the degradation condition. For example, the internal body or the external body may be degraded by supplying a degradation fluid to the respective body in the mold, for instance a heated fluid or a fluid with dissolved degradation chemicals, enzymes et cetera.

In a further aspect of the invention the mold comprises or is provided with support means for supporting at least part of the external body of the mold. The support means may be formed by an outer layer of the external body which is of non-fugitive material, i.e. is not degradable under the circumstances where the fugitive material of the internal body and/or inner layer of the external body is degraded. The support means may also be completely separate from the external body. For instance the support means may comprise a support body, for example a support container, defining a chamber in which the external body and internal body of the mold are placed for forming a 3D cell tissue structure therein. Preferably the support means, for example the support container, stays intact throughout a process of forming a 3D cell tissue structure using the mold, e.g. during the culture process. The support means, e.g. support container, may define a chamber for placing the external body and internal body of the mold therein, which chamber is sealed off or at least almost liquid-tight closed from an outside environment. Particularly the chamber defined by the support means constitutes a sterile environment in which a 3D cell tissue structure may be formed and maintained. Preferably the support means, e.g. support container, allows for a 3D cell tissue structure to be maintained in the container after degradation of at least the internal body and/or the external body of the mold. The support means, e.g. the support container, may be a part of the mold, for example be comprised in the external body of the mold, or may be an additional entity, separate from the mold, for instance a separate container made of for example a plastic. Preferably the support means, e.g. support container, is transparent to allow a visual inspection of the chamber and its contents, e.g. mold and/or supported cell structure and/or cell tissue structure. Preferably the mold according to the invention allows for the 3D cell tissue structure, for example organoid, to be cultured and analyzed in the mold after formation of the tissue structure and optionally after removal by degradation of the internal and/or external body of the mold. For example the support means may realize this, by supporting the 3D cell tissue structure after degradation of the internal and/or external body of the mold. Particularly this obviates the need to further handle or transport the formed 3D cell tissue structure. Accordingly, the handling steps and/or the handling time of the 3D cell tissue structure may be reduced with the mold according to the invention. Furthermore, a risk of problems arising due to handling such generally sensitive tissue, e.g. breaking or adversely affecting the tissue, is significantly reduced.

In a further preferred embodiment the mold according to the invention is characterized in that the internal body is made in an irregular shape, and/or any other desired shape, particularly a ventricle-like or atrium-like or heart-like shape. As the present invention relies on the internal body of the mold being degradable, such internal body can be removed out of the formed cell structure or cell tissue and the external body independent from any shape of the bodies. Thus the cell tissue that can be prepared with a mold and method of the present invention can have any desired shape, be it a regular shape or a more complex shape. Accordingly, it is possible to prepare a 3D cell tissue structure resembling any organoid, such as for example a human heart, which allows realistic modelling of a human heart functioning.

In a further preferred embodiment the mold according to the invention is characterized in that the external body comprises at least one opening in fluid communication with the cavity. The at least one opening provides an access through the external body to the cavity so that for instance cells, other constituents for the cell tissue and media can be introduced into the culture chamber.

In a further preferred embodiment the mold according to the invention is characterized in that the external body comprises a first opening in fluid communication with the cavity for receiving there through a first conduit for guiding fluid to the cavity, and a second opening in fluid communication with the cavity for receiving there through a second conduit for guiding fluid away from the cavity. The openings and respective conduits allow flow of fluid, for example cell media, to and from the cavity. Particularly the flow of fluid may be realized as a continuous flow to maintain suitable conditions for the cell tissue in the culture chamber.

In a further preferred embodiment the mold according to the invention is characterized in that the external body comprises an inlet wall in which inlet wall both the first and second opening are provided. By providing the openings in a same wall of the external body, preferably an upper wall of the external body, the other walls thereof, having no openings, may provide a fluid-tightness of that part of the external body. As such there is a reduced risk of leakage when in use of the mold.

In some embodiments, the cells that are provided to the culture chamber to form the cell tissue are progenitor cells and/or stem cells, preferably pluripotent stem cells. Non-limiting examples are somatic stem cells and induced pluripotent stem cells. In some preferred embodiments, more differentiated cells are provided to the culture chamber. In some embodiments, said differentiated cells are selected from the group consisting of cardiomyocytes, endothelial cells, fibroblasts, endocardial cells, epicardial cells, endocardial cells, smooth muscle cells, percicytes and nerve cells. In some embodiments said differentiated cells are obtained in vitro from stem cells and/or progenitor cells, using culture conditions that direct the differentiation of the stem cells and/or progenitor cells into one or more cell types of interest. Preferably, human stem cells are used, more preferably human pluripotent stem cells (hPSC). In some embodiments, said human stem cells are human induced pluripotent stem cells (hiPSC). Current technologies enable differentiation of human pluripotent stem cells into most (if not all) cell types of the human body, including the cardiovascular lineage. For instance, differentiation of hPSC to atrial cardiomyocytes (Devalla et al., 2015), ventricular cardiomyocytes (Elliott et al., 2011), pro-epicardial cells (Guadix et al., 2017), cardiac fibroblasts (Witty et al., 2014), smooth muscle cells (Witty et al., 2014) and endothelial cells (Orlova et al., 2014) has been accomplished. Human pluripotent stem cells are for instance differentiated to the cardiovascular lineage by use of a BPEL medium (Bovine Serum Albumin [BSA] Polyvinyl alcohol Essential Lipids) according to (Ng, Davis, Stanley, & Elefanty, 2008), supplemented with a mixture of cytokines (BMP4, ACTIVIN A, and a GSK3 inhibitor (Chir99021) and by administration of a Wnt inhibitor (XAV939). As another example, it is possible to differentiate hPSC to renal cells (Takasato et al., 2014), to lung tissue (Jacob et al., 2017), to gut tissue (Spence et al., 2011), to bladder cells (Oottamasathien et al., 2007) and to retinal tissue (Osakada, Ikeda, Sasai, & Takahashi, 2009).

The cells that are provided to the culture chamber are preferably present in a culture medium, liquid, gelatinous composition or cell suspension.

In preferred embodiments, said culture medium, liquid, gelatinous composition or cell suspension comprises one or more compounds for increasing the viscosity of the culture medium, liquid, gelatinous composition or cell suspension. Such compound that is able to increase the viscosity of the culture medium, liquid, gelatinous composition or cell suspension is also referred to herein as a viscosity-increasing compound. An increased viscosity of the culture medium, liquid, gelatinous composition or cell suspension facilitates tissue formation because a higher viscosity provides more support for the cultured cells. In some preferred embodiments, a compound is used that is capable of hydrogel formation in said culture medium, liquid, gelatinous composition or cell suspension. Such compound is also referred to herein as a hydrogel-forming compound. The resulting hydrogel forms a three-dimensional network structure, preferably in the presence of, or together with, the culture medium, liquid, gelatinous composition or cell suspension. The presence of a hydrogel particularly facilitates tissue formation because its 3D structure provides more support for the cultured cells in the 3D space. Said viscosity-increasing compound or hydrogel-forming compound for instance comprises a compound that can be polymerized and/or crosslinked. As used herein, the term “polymerization” means that a monomer is coupled to another monomer or to a growing polymer chain, resulting in (larger) polymer chains. The term “crosslinking” refers to the formation of at least one bond between at least two different polymer chains. Said bond, which is typically formed between two branched polymer chains, is preferably a covalent bond or ionic bond. Polymerization and/or crosslinking of a viscosity-increasing compound in said culture medium, liquid, gelatinous composition or cell suspension typically results in a culture medium, liquid, gelatinous composition or cell suspension with a higher viscosity. Polymerization and/or crosslinking of a hydrogel-forming compound in said culture medium, liquid, gelatinous composition or cell suspension typically results in a hydrogel that at least in part contains the culture medium, liquid, gelatinous composition or cell suspension and that can encapsulate the cells. Preferably a colloidal or polymer network is formed that does not change its shape when in the steady state. This provides the advantage that the hydrogel provides particularly good support for the cultured cells, so that the supported cell structure can also maintain its shape when the internal body is degraded. This way, a hollow cavity surrounded by a supported cell structure is formed, which can be further cultured in order to grow a hollow tissue such as an organoid. In preferred embodiments, said viscosity-increasing compound or said hydrogel-forming compound comprises branched polymers. Non-limiting examples of viscosity-increasing compounds and/or hydrogel-forming compounds are fibrinogen, collagen type I, polyvinyl alcohol, calcium-alginate, Pluronic F-127, gelatin, hyaluronic acid, dextran, chitosan, Matrigel and Polyethylene glycol.

In preferred embodiments of the invention, a viscosity-increasing compound or a hydrogel-forming compound is used that is degraded after sufficient cell tissue has formed. The degradation of said viscosity-increasing compound or said hydrogel-forming compound should not completely or significantly disrupt the structure and the functionality of the cell tissue. Preferably, the degradation of said viscosity-increasing compound or said hydrogel-forming compound does not adversely affect the structure and the functionality of the cell tissue.

In particularly preferred embodiments, a method according to the invention is provided wherein fibrinogen and thrombin are present in the culture medium, liquid, gelatinous composition or cell suspension. In the presence of thrombin, fibrinogen typically forms fibrin strands that polymerize and are crosslinked with other fibrin strands to form an extensive interconnected fibrin network. Fibrinogen is thus used in these embodiments as a hydrogel-forming compound. The resulting fibrin network provides a suitable support for the cultured cells, thereby forming a supported cell structure. Subsequently, the fibrin network is degraded, preferably by metalloproteases produced by the cells. The fibrin network is typically degraded within several days without adversely affecting the structure and the functionality of the cell tissue that is being formed. This time frame of several days is sufficiently long to enable the formation of cell tissue around the hollow structure (cavity) that is present after degradation of the internal body.

In order to promote cell tissue formation, a culture medium is preferably used that comprises nutrients and growth factors. In some embodiments, said culture medium comprises serum. Preferably, oxygen is provided to the cells as well, for instance by placing the mold in a culture incubator at 37 degrees Celsius, 5% CO₂ and 21% O₂.

In some embodiments, the cultured cells form themselves a three-dimensional network structure, either in the presence or absence of a viscosity-increasing compound or a hydrogel-forming compound. In some embodiments, the cells form a three-dimensional network by cell self-assembly. In some embodiments, cells are used that comprise a non-natural compound that enables the coupling of the cells. In some embodiments, cells are used that comprise bio-orthogonal groups on their cell membranes, which groups can bind each other, thereby linking cells to each other. Such links, which are typically referred to as “click-bio-orthogonal chemistry” (Rogozhnikov, O'Brien, Elahipanah, & Yousaf, 2016), are suitable for binding the membranes of cells, thereby binding the cells to each other. This way, a 3D cellular network structure is formed that provides support for cultured cells and facilitates tissue formation. In some embodiments, several cells are provided with ketone groups on their cell surface while other cells are provided with oxyamine groups on their cell surface. Subsequently, upon mixing these cells, the ketone groups and the oxyamine groups will bind each other, thereby binding the cells to each other.

In some preferred embodiments, a method according to the present invention is used for preparing a cardiac organoid.

As described herein before, a major limitation in better understanding the underlying mechanisms of cardiac disease as well as in avoiding drug-induced cardiotoxicity is the lack of predictable models of the human heart. In past years new procedures have been developed to generate more predictive 3D hPSC-cardiomyocyte based tissues, such as biowire (Nunes et al., 2013) and engineered heart tissue (EHT) (Schaaf et al., 2011). Although these cardiac tissues allow the measurement of contraction force, their 3D structure is limited to cardiac tissue strips and therefore cannot directly model the heart's fluid pumping function, as the cardiac tissue strips do not allow measurements of cardiac volume and fluidic pressure.

Although these cardiac tissue strips hold great promise in the field of cardiac disease modeling, they have the important disadvantage of lacking the pumping functionality of a human heart. Other known techniques that use molds to form 3D cell tissue structures, such as the use of a balloon and catheter as described in WO 2015/184273, are limited to 3D cell tissue structures with rather simple and straightforward shapes. Mimicking a 3D pumping heart function is preferred, for instance to analyze pumping function (ejection fraction) and liquid flow, comparable to pumping blood through the circulation by an in vivo heart. Moreover, the fact that cardiac output, ejection fraction and cardiac electrophysiology are main clinical diagnostic outputs underlines the importance of being able to measure these cardiac parameters in in vitro cardiac (disease) models. Preferably, a human cardiac organoid is produced. In contrast to the known techniques, the present invention now enables the generation of hollow cardiac structures with a comparable shape as the cavities present in the human heart, where the four corresponding cavities, two atrium and two ventricles, can be molded and formed to scale, and are capable of pumping fluid similar to the pumping of blood by the human heart. Particularly the present invention allows separate inlet and outlet channels to be provided in the 3D cell tissue structures, to enable a continuous flow of liquid through the cavities and channels of the tissue structure, for instance by pumping fluid received via an inlet channel in the tissue structure out of the tissue structure via an outlet channel which is physically separated from the inlet channel. This more closely mimics a human heart as compared to the known techniques that at best provide a 3D cell tissue structure with rather simple and straightforward shape and a single inlet/outlet channel.

Methods according to the present invention for preparing a cardiac organoid amongst other things provide the advantages that a hollow cardiac structure can be formed without the need of additional handling steps of moving cardiac tissue from one vessel or container to another and that the hollow structure can be in many different, if desired irregular, forms.

According to some embodiments of the present invention, a cardiac organoid, preferably a human cardiac organoid, is prepared by:
preparing an external body having an inner surface delimiting a cavity in the external body;
preparing an internal body of fugitive material in a predefined shape and having an outer surface;
placing the internal body inside the cavity of the external body, wherein the inner surface and outer surface define a culture chamber there between;
providing cells, particularly stem cells, progenitor cells, cardiomyocytes and/or other cardiac cells into the culture chamber;
subjecting the cells to incubating conditions for a period of time to promote the formation of a supported cell structure, and/or to incubation conditions to promote the formation of cell tissue, in the culture chamber around the outer surface of the internal body;
subjecting the internal body of fugitive material to a condition whereto the fugitive material reacts thereby degrading the internal body for removal from the cavity without significantly affecting an integrity of a formed cell structure or cell tissue in the culture chamber and maintaining a functioning of the formed cell structure or cell tissue.

The step of promoting the formation of a supported cell structure preferably comprises the encapsulation of the cells into a hydrogel.

The step of subjecting the internal body to a condition whereto the fugitive material reacts preferably comprises liquefying and/or dissolving of the internal body by a change in temperature, particularly dissolving the internal body by increasing the temperature from a start temperature below 10 degrees Celsius to a degradation temperature between 10 and 40 degrees Celsius.

In a particular embodiment of the method of the present invention the internal body is liquefied and/or dissolved by supplying a liquefying or dissolving agent to the internal body.

In a further particular embodiment of the method of the present invention said liquefying or dissolving agent comprises an enzyme or chemical compound or composition.

In another embodiment of the method of the present invention the internal body is liquefied and/or dissolved by sonication or by exposure to light at a certain wavelength.

Particularly, in a further embodiment of the method of the present invention the step of providing cells in the culture chamber comprises providing cells in the cavity of the external body followed by placing of the internal body inside the cavity of the external body to position the cells, preferably stem cells, progenitor cells, cardiomyocytes and/or other cardiac cells, in the culture chamber.

In a further particular embodiment of the method of the present invention the external body is prepared by preparing a first part of the external body comprising a first section of the cavity, preparing a separate second part of the external body comprising a second section of the cavity, and coupling of the first part and second part to each other such that the first section of the cavity and the second section of the cavity communicate with each other thereby forming the cavity. As such the external body may be prepared from two initially separate parts, for example halves, allowing for easy introduction and positioning of the internal body inside the cavity.

Said cells that are provided to the culture chamber are preferably stem cells, progenitor cells, cardiomyocytes and/or other cardiac cells.

In some embodiments the cell tissue formation precedes the step of degrading the internal body. In other embodiments cell tissue is formed after degradation of the internal body. In such cases, a supported cell structure is formed first, for instance by encapsulation of cells into a hydrogel, before the internal body is degraded so that the cells remain around the cavity that is formed after degradation of the internal body.

Some embodiments provide a method for preparing a hollow 3D cell tissue structure such as an organoid, in particular a human heart mimic, comprising the steps of:
preparing an external body having an inner surface delimiting a cavity in the external body;
preparing an internal body of fugitive material in a predefined shape and having an outer surface;
placing the internal body inside the cavity of the external body, wherein the inner surface and outer surface define a culture chamber there between;
providing cells, particularly stem cells, progenitor cells, cardiomyocytes and/or other cardiac cells into the culture chamber;
subjecting the cells to incubating conditions for a period of time to promote the formation of a supported cell structure in the culture chamber around the outer surface of the internal body;
subjecting the internal body of fugitive material to a condition whereto the fugitive material reacts thereby degrading the internal body for removal from the cavity without significantly affecting an integrity of the supported cell structure; and
allowing the formation of a hollow 3D cell tissue.

As stated herein before, said cells are preferably present in a culture medium, liquid, gelatinous composition or cell suspension that comprises a viscosity-increasing compound in order to increase support for the cultured cells. In particularly preferred embodiments, said cells are present in a culture medium, liquid, gelatinous composition or cell suspension that comprises a hydrogel-forming compound, so that a three-dimensional network is formed that provides a suitable support for the cells. In a preferred embodiment, the internal body is made of gelatin that can be degraded by a temperature above 25° C.

Some embodiments provide a method for preparing a cardiac organoid, preferably a human cardiac organoid, comprising the steps of:
preparing an external body having an inner surface delimiting a cavity in the external body;
preparing an internal body in a predefined shape and having an outer surface, wherein said internal body is made of gelatin;
placing the internal body inside the cavity of the external body, wherein the inner surface and outer surface define a culture chamber there between;
providing stem cells, progenitor cells, cardiomyocytes and/or other cardiac cells into the culture chamber, wherein the cells are present in a culture medium, liquid, gelatinous composition or cell suspension that comprises a viscosity-increasing compound and/or a hydrogel-forming compound;
allowing for an increase of viscosity of the culture medium, liquid, gelatinous composition or cell suspension, and/or the formation of a hydrogel, thereby forming a supported cell structure;
liquefying and/or dissolving the gelatin internal body at a temperature of between 20 and 40° C.; and
subjecting the cells to incubating conditions for a period of time to promote cardiac tissue formation.

Said gelatin internal body is preferably liquefied and/or dissolved at a temperature of between 25 and 40° C., more preferably at a temperature of between 30 and 40° C., more preferably at a temperature of between 35-37° C., more preferably at a temperature of about 37° C.

Said culture medium, liquid, gelatinous composition or cell suspension preferably comprises fibrinogen and thrombin, which allows the formation of a fibrin network that provides a suitable support for the cultured cells and that can subsequently be degraded, preferably by metalloproteases produced by the cultured cells, without adversely affecting the structure and the functionality of the cell tissue that is being formed.

Some embodiments therefore provide a method for preparing a cardiac organoid, preferably a human cardiac organoid, comprising the steps of:
preparing an external body having an inner surface delimiting a cavity in the external body;
preparing an internal body in a predefined shape and having an outer surface, wherein said internal body is made of gelatin;
placing the internal body inside the cavity of the external body, wherein the inner surface and outer surface define a culture chamber there between;
providing stem cells, progenitor cells, cardiomyocytes and/or other cardiac cells into the culture chamber, wherein the cells are present in a culture medium, liquid, gelatinous composition or cell suspension that comprises fibrinogen and thrombin;
allowing for polymerization of the fibrinogen, thereby forming a supported cell structure;
liquefying and/or dissolving the gelatin internal body at a temperature of between 20 and 40° C.; and
subjecting the cells to incubating conditions for a period of time to promote cardiac tissue formation.

Some embodiments provide a method for preparing a cardiac organoid, preferably a human cardiac organoid, comprising the steps of:
preparing an external body having an inner surface delimiting a cavity in the external body;
preparing an internal body in a predefined shape and having an outer surface, wherein said internal body is made of gelatin;
placing the internal body inside the cavity of the external body, wherein the inner surface and outer surface define a culture chamber there between;
providing stem cells, progenitor cells, cardiomyocytes and/or other cardiac cells into the culture chamber, wherein the cells are present in a culture medium, liquid, gelatinous composition or cell suspension that comprises fibrinogen and thrombin;
allowing for polymerization of the fibrinogen, thereby forming a supported cell structure;
liquefying and/or dissolving the gelatin internal body at a temperature of between 20 and 40° C.; and
allowing the formation of a cardiac organoid.

In some embodiments, the recited steps of the methods according to the invention are consecutive steps.

A use of a mold according to the present invention for preparing a hollow 3D cell tissue structure, preferably an organoid with a cavity, is also provided herewith. In some preferred embodiments, said mold is used for the preparation of a heart mimic. Some embodiments therefore provide a use of a mold according to the invention for the preparation of a cardiac organoid. Said cardiac organoid is preferably a human cardiac organoid. In order to prepare a hollow 3D cell tissue structure, such as for instance a cardiac organoid, it is preferred to provide cells, particularly stem cells, progenitor cells, cardiomyocytes and/or other cardiac cells, to the culture chamber. Further provided is therefore a use of:
a mold according to the present invention; and
cells, preferably stem cells, progenitor cells, cardiomyocytes and/or other cardiac cells;
for the preparation of a hollow 3D cell tissue structure, preferably an organoid with a cavity, more preferably a cardiac organoid.

As stated above, said cells are preferably present in a culture medium, liquid, gelatinous composition or cell suspension that comprises a viscosity-increasing compound and/or a hydrogel-forming compound in order to provide a suitable support for the cells. In some embodiments, said culture medium, liquid, gelatinous composition or cell suspension comprises fibrinogen and thrombin. In order to prepare a cardiac organoid, the internal body is preferably of a ventricle-like shape or atrium-like shape or heart-like shape. In some preferred embodiments, the internal body is made of gelatin that can be degraded by a temperature above 25° C.

Also provided is a mold according to the invention that has been provided with cells of interest. Such cell-comprising mold according to the invention, also referred to herein as a bioreactor according to the invention, allows the formation of a hollow 3D cell tissue structure such as for instance an organoid with a cavity.

Further provided is therefore a bioreactor comprising a mold according to the present invention and cells that are present in the culture chamber of said mold, between the inner surface of the external body and the outer surface of the internal body. Some embodiments provide a bioreactor for preparing a hollow 3D cell tissue structure such as an organoid, in particular a human heart mimic, comprising: an external body having an inner surface delimiting a cavity in the external body; an internal body having an outer surface, wherein the internal body is positioned in the cavity of the external body, wherein the inner surface of the external body and the outer surface of the internal body define a culture chamber there between and wherein cells are present in said culture chamber, characterized in that the internal body is made in a predefined shape of a fugitive material to enable degradation of the internal body without significantly affecting an integrity of a formed cell structure and/or cell tissue. In some embodiments the bioreactor of the present invention comprises a support means, e.g. a support container, as described herein, which support means is configured to support at least the 3D cell structure and/or cell tissue after degradation of the internal and/or external body of the mold.

As described herein before, preferably both the internal body and the external body are made of a fugitive material. In some embodiments, the internal body and the external body are formed from a different fugitive material.

The fugitive material is preferably adapted to degrade above or below a temperature threshold value and/or in the presence of an enzyme and/or in the presence of a reactive chemical compound and/or by sonication and/or in the presence of light with a wavelength above or below a threshold value. In some embodiments, the fugitive material is adapted to degrade at a temperature between 10-50 degrees Celsius, more particularly between 25-40 degrees Celsius. In some embodiments, the fugitive material is substantially solid below 10 degrees Celsius and is adapted to dissolve and/or liquefy above 10 degrees Celsius, in particular to dissolve and/or liquefy at a temperature between 10-42 degrees Celsius. In some embodiments, the fugitive material is substantially solid above 15 degrees Celsius and is adapted to dissolve and/or liquefy below 15 degrees Celsius. The fugitive material preferably comprises gelatin, particularly at least 10% gelatin, and/or Pluronic® F-127 and/or poly(vinyl alcohol) (PVA).

Some particular embodiments thus provide a method for preparing a hollow 3D cell tissue structure such as an organoid, in particular a human heart mimic, comprising the steps, particularly consecutive steps, of:
preparing an external body having an inner surface delimiting a cavity in the external body;
preparing an internal body of degradable material in a predefined shape and having an outer surface;
placing the internal body inside the cavity of the external body, wherein the inner surface and outer surface define a culture chamber there between;
providing cells, particularly stem cells, progenitor cells, cardiomyocytes and/or other cardiac cells into the culture chamber;
subjecting the cells to incubating conditions for a period of time to promote the formation of a supported cell structure, and/or to incubating conditions to promote the formation of a cell tissue, in the culture chamber around the outer surface of the internal body;
subjecting the internal body of degradable material to a condition whereto the degradable material reacts thereby degrading the internal body for removal from the cavity without significantly affecting an integrity of a formed supported cell structure or cell tissue in the culture chamber and maintaining a functioning of the supported cell structure or cell tissue. In some embodiments, said method comprises a further step of allowing the formation of a hollow 3D tissue. Some particular embodiments provide a bioreactor for preparing a hollow 3D cell tissue structure such as an organoid, in particular a human heart mimic, comprising: an external body having an inner surface delimiting a cavity in the external body; an internal body having an outer surface, wherein the internal body is positioned in the cavity of the external body, wherein the inner surface of the external body and the outer surface of the internal body define a culture chamber there between and wherein cells are present in said culture chamber, characterized in that the internal body is made in a predefined shape of a degradable material to enable degradation of the internal body without significantly affecting an integrity of a formed cell structure and/or cell tissue.

Some embodiments provide a method or bioreactor according to the invention wherein the degradable material is degradable above or below a temperature threshold value and/or in the presence of an enzyme and/or in the presence of a reactive chemical compound and/or by sonication and/or in the presence of light with a wavelength above or below a threshold value. Some embodiments provide a method or bioreactor according to the invention wherein the degradable material is degradable at a temperature between 10-50 degrees Celsius, preferably between 25-40 degrees Celsius. Some embodiments provide a method or bioreactor according to the invention wherein the degradable material is substantially solid below 10 degrees Celsius and dissolves and/or liquefies above 10 degrees Celsius, preferably dissolves and/or liquefies at a temperature between 10-42 degrees Celsius. Some embodiments provide a method or bioreactor according to the invention wherein the degradable material is substantially solid above 15 degrees Celsius and dissolves and/or liquefies below 15 degrees Celsius. Some embodiments provide a method according to the invention wherein the step of subjecting the internal body to a condition whereto the degradable material reacts comprises liquefying and/or dissolving of the internal body by a change in temperature, preferably comprises dissolving the internal body by increasing the temperature from a start temperature below 10 degrees Celsius to a degradation temperature between 10 and 40 degrees Celsius. Some embodiments provide a method according to the invention wherein the internal body is liquefied and/or dissolved by supplying a liquefying or dissolving agent, preferably an enzyme or chemical compound or composition, to the internal body. Some embodiments provide a method according to the invention wherein the internal body is liquefied and/or dissolved by sonication and/or in the presence of light with a wavelength above or below a threshold value.

In some embodiments, the internal body is made in an irregular shape, and/or any other desired shape, particularly a ventricle-like or atrium-like or heart-like shape. In some embodiments, the external body comprises at least one opening in fluid communication with the cavity. The external body preferably comprises a first opening in fluid communication with the cavity for receiving there through a first conduit for guiding fluid to the cavity, and a second opening in fluid communication with the cavity for receiving there through a second conduit for guiding fluid away from the cavity. In some embodiments, the external body comprises an inlet wall in which inlet wall both the first and second opening are provided.

Further provided is a hollow 3D cell tissue structure obtainable or obtained by a method according to the present invention. Said hollow 3D cell tissue structure is preferably an organoid, more preferably an organoid with a cavity such as for instance a cardiac organoid, a renal organoid, a lung organoid, a gut organoid, a bladder organoid or an eye organoids. In some preferred embodiments, said organoid is a human cardiac organoid.

An organoid produced by a method according to the present invention is amongst other things useful for drug discovery and development. For instance, with the methods according to the present invention, cardiac organoids can be made that closely mimic (human) heart's cavities (atrium and/or ventricles) and that are capable of pumping fluid, and that are therefore a preferred tool for drug discovery and development.

Other applications of an organoid according to the present invention are in the field of regenerative medicine. Organoids can be prepared in vitro using the methods according to the present invention. Subsequently, such organoid or a part thereof can be implanted into an individual. The use of organoid tissue prepared from cells of a given individual significantly diminishes the chance of organ transplant rejection.

These and other objects and aspects of the present invention are hereinafter further elucidated by the appended drawing and the corresponding embodiment, which forms part of the present application. The drawing is not in any way meant to reflect a limitation of the scope of the invention, unless this is clearly and explicitly indicated. In the drawing:

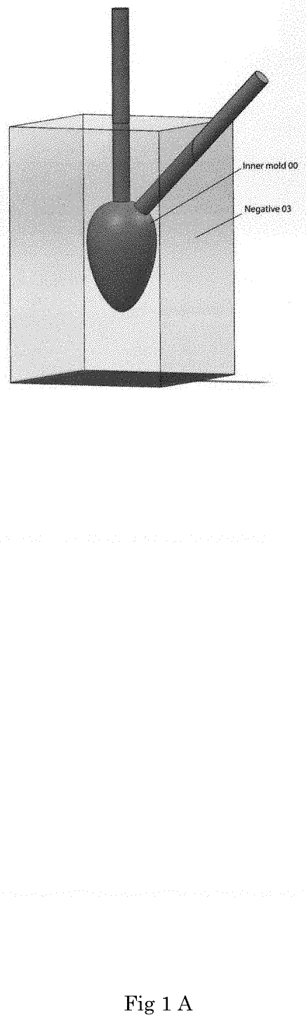

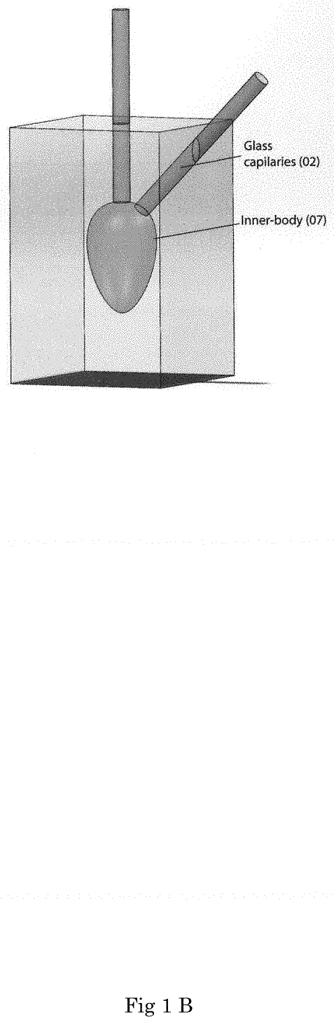

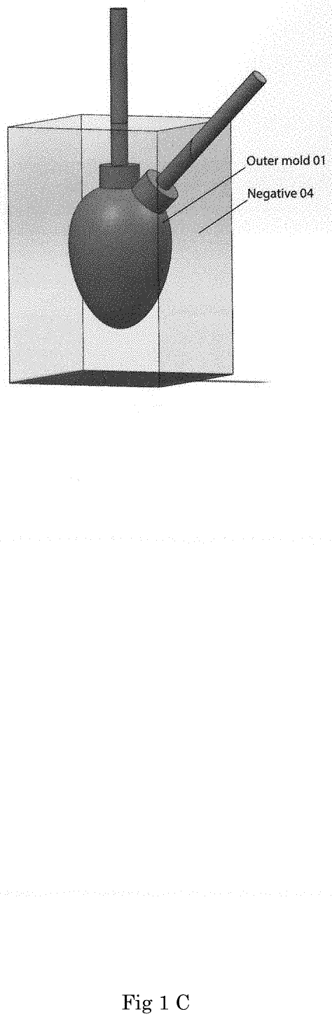

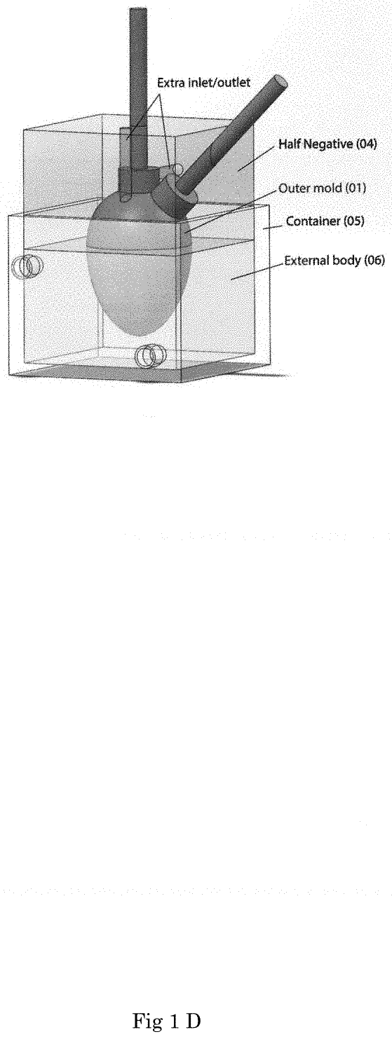

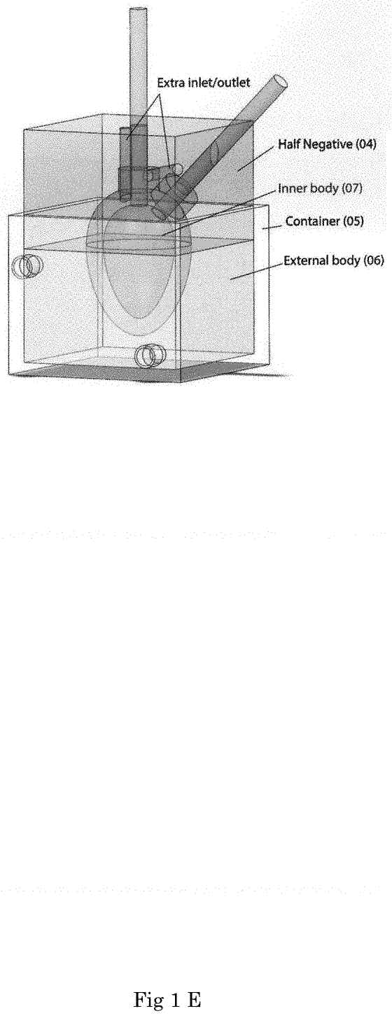

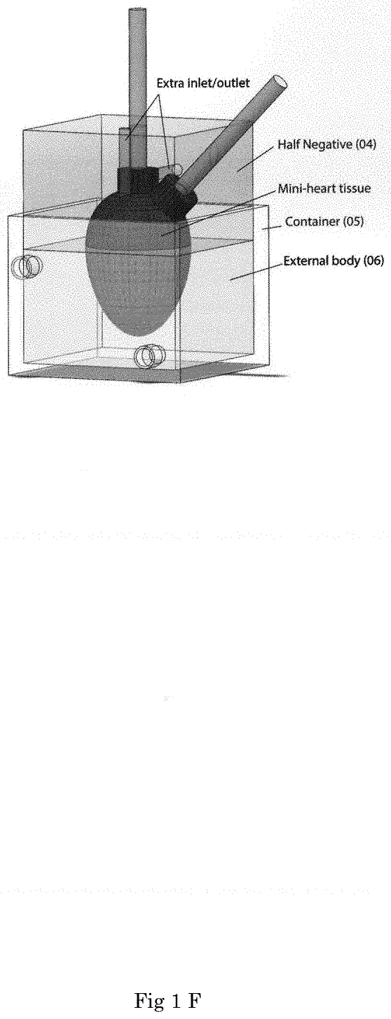

FIG. 1A-1F show consecutive steps of the preparation of an embodiment of a mold and bioreactor according to the invention.

In this application similar or corresponding features are denoted by similar or corresponding reference signs. The description of the various embodiments is not limited to the example shown in the figures and the reference numbers used in the detailed description and the claims are not intended to limit the description of the embodiments, but are included to elucidate the embodiments by referring to the example shown in the figures.

In an embodiment as shown in FIG. 1, the invention concerns a mold comprising an internal body inside the cavity of an external body, which mold is formed as follows. In a first step an inner-mold (00) is 3D printed in a desired predetermined shape, herein in the shape of a human heart mimic with separate inlet channel and outlet channel, using a biocompatible resin. The inner-mold (00) is used to cast a negative (03) of the inner-mold (00) in polydimethylsiloxane as shown in FIG. 1A. The negative (03) is then cut in half (not shown), for instance by using a sharp blade to allow the inner-mold (00) to be removed from the inside of the negative (03). Next the two halves of the negative (03) are coupled together again to form the negative (03) with an empty cavity formed by the removed inner-mold (00). The two halves of negative (03) can be tightly clamped together in order to make the cavity at least substantially liquid-tight so as to avoid leakage of a liquid introduced into the cavity. A glass capillary (02) is inserted through each of the inlet and outlet spaces formed in the negative (03) as a result of the inner-mold (00) comprising respective inlet outlet channels. Each of the glass capillaries extends with an end into the human-heart shaped cavity of negative (03).

Gelatin (Sigma-Aldrich) is dissolved as 10% (w/v) in PBS or medium and autoclaved at 120 degrees Celsius. The gelatin solution is stored at 4 degrees Celsius. The stored gelatin solution is warmed to 37 degrees Celsius and is pipetted into the negative mold (03) through one of the glass capillaries (02) until the cavity of negative mold (03) is filled with gelatin. The negative mold (03) filled with gelatin is cooled to 4 degree Celsius in order to gelate the gelatin. The gelated gelatin forms a human-heart shaped inner body (07) of the mold according to the invention, as shown in FIG. 1B.

In a next step an outer-mold (01) is 3D printed using a biocompatible resin in a shape corresponding to the inner-mold (00) but larger in size. The outer-mold (01) is used to cast an outer negative (04) of outer-mold (01) in polydimethylsiloxane as shown in FIG. 1C. The outer negative (04) is then cut in half, for example using a sharp blade (not shown). This allows the outer-mold (01) to be removed from the inside of the outer negative (04). Optionally, the outer-mold (01) may be kept inside a part of the cut negative (04). However, a bottom part of the negative (04) is separated from a top part of the negative (04). The top part of the negative (04) together with the outer-mold (01), or optionally the outer-mold (01) alone, is then placed inside a cavity defined by support means, i.e. support container (05), with the cavity in the container (05) being sealed from an outside environment as shown in FIG. 1D due to tight fitting of the top part of negative (04) over container (05). Container (05) contains a pair of inlet/outlet cannulas that gives access to its interior independently of the inlet/outlets of the inner-body (07) and/or the tissue.

In order to prepare the external body, the stored gelatin solution previously described is warmed to 37 degrees Celsius and is introduced into the remaining free space in the cavity of the container (05) not occupied by the outer mold (01) through the inlet/outlet cannulas. The assembly of container (05), the top part of the negative (04), the outer-mold (01) and the introduced gelatin is cooled at 4 degrees Celsius in order to gelate the gelatin. After gelatin gelation the top part of the negative (04) and the outer-mold (01) are removed from the container (05), thereby leaving a gelatin external body (06) of the mold according to the invention behind inside the container (05).

The outer-mold (01) is next removed from the top part of the negative (04). An extra pair of inlet and outlet are made on negative (04) from an outside surface into a lumen surface.

Next, the negative (03) is opened and the solid inner-body (07) of gelatin is removed together with the inlet and outlet capillaries. The inner-body (07) is then partly placed in the space defined by the external body (06) in the container (05) and appropriately aligned therewith using the inlet and outlet capillaries. The external body (06) together with the inner-body (07) are thus positioned inside the container (05), as shown in FIG. 1E. The appropriate alignment is possible through the perfect match of the external body (06) and the inner body (07), creating a uniform gap between an outer surface of the inner-body (07) and an inner surface of the external body (06).

Beating cardiomyocytes are prepared as follows. Human embryonic stem cells (hESC) and/or human induced pluripotent stem cells (hiPSC) coming from in vitro cell cultures or from commercial available sources are cultured on Vitronectin Recombinant Human Protein (Life technologies) coated plastic plates in E8 medium (Life Technologies). The hESC and hiPSC are passaged using PBS (Life Technologies) containing EDTA 0.5 mM (Life Technologies) or TryplE (Gibco).

Differentiation into the cardiac lineage is induced in a monolayer as described previously (Elliott et al., 2011; (van den Berg, Elliott, Braam, Mummery, & Davis, 2016). Briefly, 25×103/cm2 are seeded on plates coated with 75 μg/mL (growth factor reduced) Matrigel (Corning) the day before differentiation (day −1). At day 0, cardiac mesoderm is induced by changing E8 to BPEL medium (Bovine Serum Albumin [BSA] Polyvinyl alcohol Essential Lipids; (Ng et al., 2008)), supplemented with a mixture of cytokines (20 ng/mL BMP4, R&D Systems; 20 ng/mL ACTIVIN A, Miltenyi Biotec; 1.5 μM GSK3 inhibitor CHIR99021, Axon Medchem). After 3 days, cytokines are removed and a Wnt inhibitor (5 μM, XAV939, Tocris Bioscience) is added for 3 days. BPEL medium is refreshed every 3-4 days.

To generate the cardiac tissue the beating cardiomyocytes are dissociated using TryplE 1× for 10 mins and resuspended in cell culture medium with 10% Serum (e.g. 1×DMEM with 10% fetal calf serum).

The resuspended cells are mixed at a final density of 5×10⁶ to 20×10⁶ cells/ml with 2 to 5 mg/ml bovine fibrinogen (stock solution: 200 mg/ml plus aprotinin 0.5 μg/mg fibrinogen in NaCl 0.9%, Sigma F4753), 100 μl/ml Matrigel (BD Bioscience 356235). To the final mix thrombin is added at 1:300 (100 U/ml, Sigma Aldrich T7513), resuspended well and the entire solution is pipetted into the gap between the inner body (07) and the external body (06) using the inlet existing on the negative (04). A thus formed bioreactor comprising the container (05) plus inner body and external body of the mold and the cell suspension, as shown in FIG. 1F, is kept at 4 degrees Celsius during the following procedure. The inlet is closed with a stopper and the bioreactor is placed in the fridge for 30 mins for polymerization of the fibrinogen.

After fibrinogen polymerization the bioreactor is transferred into a 37° Celsius, 5% CO₂ humidified cell culture incubator in order to degrade the inner body (07) and the external body (06) by melting the gelatin. After the gelatin has melted a free standing tissue made of cells and fibrinogen remains inside the bioreactor. The inside of the tissue is perfused with medium at a flow rate of 100 ul per minute for 10 mins. Hence, the gelatin is removed by flushing the inner cavity of the tissue with medium. Furthermore, inlet and outlet of the tissue are closed and medium is perfused via the inlet/outlet cannulas present on the container (05) at a flow rate of 100 ul per hour. The tissue starts contracting 3 to 4 days after the making.

While the current application may describe features as part of the same embodiment or as parts of separate embodiments, the scope of the present invention also includes embodiments comprising any combination of all or some of the features described herein.

As used herein, the terms “in particular”, “particularly” and “preferably” are used interchangeably.

### REFERENCES

Devalla, H. D., Schwach, V., Ford, J. W., Milnes, J. T., El-Haou, S., Jackson, C., . . . Passier, R. (2015). Atrial-like cardiomyocytes from human pluripotent stem cells are a robust preclinical model for assessing atrial-selective pharmacology. EMBO Molecular Medicine, 7(4), 394-410. http://doi.org/10.15252/emmm.201404757
Elliott, D. A., Braam, S. R., Koutsis, K., Ng, E. S., Jenny, R., Lagerqvist, E. L., . . . Stanley, E. G. (2011). NKX2-5eGFP/w hESCs for isolation of human cardiac progenitors and cardiomyocytes. Nature Methods, (october), 1-7. http://doi.org/10.1038/nmeth.1740
Guadix, J. A., Orlova, V. V., Giacomelli, E., Bellin, M., Ribeiro, M. C., Mummery, C. L., . . . Passier, R. (2017). Human Pluripotent Stem Cell Differentiation into Functional Epicardial Progenitor Cells. Stem Cell Reports, 9(6), 1754-1764. http://doi.org/10.1016/j.stemcr.2017.10.023
Jacob, A., Morley, M., Hawkins, F., McCauley, K. B., Jean, J. C., Heins, H., . . . Kotton, D. N. (2017). Differentiation of Human Pluripotent Stem Cells into Functional Lung Alveolar Epithelial Cells. Cell Stem Cell, 21(4), 472-488.e10. http://doi.org/10.1016/j.stem.2017.08.014
Ng, E. S., Davis, R., Stanley, E. G., & Elefanty, A. G. (2008). A protocol describing the use of a recombinant protein-based, animal product-free medium (APEL) for human embryonic stem cell differentiation as spin embryoid bodies. Nature Protocols, 3(5), 768-76. http://doi.org/10.1038/nprot.2008.42
Nunes, S. S., Miklas, J. W., Liu, J., Aschar-Sobbi, R., Xiao, Y., Zhang, B., . . . Radisic, M. (2013). Biowire: a platform for maturation of human pluripotent stem cell-derived cardiomyocytes. Nature Methods, 10 (8), 781-7. http://doi.org/10.1038/nmeth.2524
Oottamasathien, S., Wang, Y. Q., Williams, K., Franco, O. E., Wills, M. L., Thomas, J. C., . . . Matusik, R. J. (2007). Directed differentiation of embryonic stem cells into bladder tissue. Developmental Biology, 304(2), 556-566. http://doi.org/10.1016/j.ydbio.2007.01.010
Orlova, V. V., Drabsch, Y., Freund, C., Petrus-Reurer, S., Van Den Hil, F. E., Muenthaisong, S., . . . Mummery, C. L. (2014). Functionality of endothelial cells and pericytes from human pluripotent stem cells demonstrated in cultured vascular plexus and zebrafish xenografts. Arteriosclerosis, Thrombosis, and Vascular Biology, 34(1), 177-186. http://doi.org/10.1161/ATVBAHA.113.302598
Osakada, F., Ikeda, H., Sasai, Y., & Takahashi, M. (2009). Stepwise differentiation of pluripotent stem cells into retinal cells. Nature Protocols, 4(6), 811-824. http://doi.org/10.1038/nprot.2009.51
Rogozhnikov, D., O'Brien, P. J., Elahipanah, S., & Yousaf, M. N. (2016). Scaffold Free Bio-orthogonal Assembly of 3-Dimensional Cardiac Tissue via Cell Surface Engineering. Scientific Reports, 6(December), 39806. http://doi.org/10.1038/srep39806
Schaaf, S., Shibamiya, A., Mewe, M., Eder, A., Stohr, A., Hirt, M. N., . . . Hansen, A. (2011). Human engineered heart tissue as a versatile tool in basic research and preclinical toxicology. PloS One, 6(10), e26397. http://doi.org/10.1371/journal.pone.0026397
Spence, J. R., Mayhew, C. N., Rankin, S. A., Kuhar, M., Vallance, E., Tolle, K., . . . Wells, J. M. (2011). Directed differentiation of human pluripotent stem cells into intestinal tissue in vitro. Nature, 470(7332), 105-109. http://doi.org/10.1038/nature09691.Directed
Takasato, M., Er, P. X., Becroft, M., Vanslambrouck, J. M., Stanley, E. G., Elefanty, A. G., & Little, M. H. (2014). Directing human embryonic stem cell differentiation towards a renal lineage generates a self-organizing kidney. Nature Cell Biology, 16(1), 118-126. http://doi.org/10.1038/ncb2894
van den Berg, C. W., Elliott, D. A., Braam, S. R., Mummery, C. L., & Davis, R. P. (2016). Differentiation of Human Pluripotent Stem Cells to Cardiomyocytes Under Defined Conditions. In A. Nagy & K. Turksen (Eds.), Patient-Specific Induced Pluripotent Stem Cell Models: Generation and Characterization (pp. 163-180). New York, N.Y.: Springer New York. http://doi.org/10.1007/7651_2014_178
Witty, A. D., Mihic, A., Tam, R. Y., Fisher, S. A., Mikryukov, A., Shoichet, M. S., . . . Keller, G. (2014). Generation of the epicardial lineage from human pluripotent stem cells. Nature Biotechnology, 32(10), 1026-35. http://doi.org/10.1038/nbt.3002

1. A mold for preparing a hollow 3D cell tissue structure such as an organoid, in particular a human heart mimic, comprising: an external body having an inner surface delimiting a cavity in the external body; an internal body having an outer surface, wherein the internal body is positioned in the cavity of the external body, wherein the inner surface of the external body and the outer surface of the internal body define a culture chamber there between for receiving cells therein, characterized in that the internal body is made in a predefined shape of a fugitive material to enable degradation of the internal body without significantly affecting an integrity of a formed cell structure and/or cell tissue.

2. A mold according to claim 1, characterized in that the internal body and the external body are made of a fugitive material.

3. A mold according to claim 2, characterized in that the internal body and the external body are formed from a different fugitive material.

4. A mold according to claim 3, characterized in that the fugitive material comprises gelatin, particularly at least 10% gelatin, and/or Pluronic® F-127 and/or poly(vinyl alcohol) (PVA).

5. A mold according to any one of the preceding claims, characterized in that the fugitive material is adapted to degrade above or below a temperature threshold value and/or in the presence of an enzyme and/or in the presence of a reactive chemical compound and/or by sonication and/or in the presence of light with a wavelength above or below a threshold value.

6. A mold according to claim 5, characterized in that the fugitive material is adapted to degrade at a temperature between 10-50 degrees Celsius, more particularly between 25-40 degrees Celsius.

7. A mold according to any one of the preceding claims, characterized in that the fugitive material is substantially solid below 10 degrees Celsius and is adapted to dissolve and/or liquefy above 10 degrees Celsius, in particular to dissolve and/or liquefy at a temperature between 10-42 degrees Celsius.

8. A mold according to any one of claims 1-6, characterized in that the fugitive material is substantially solid above 15 degrees Celsius and is adapted to dissolve and/or liquefy below 15 degrees Celsius.

9. A mold according to any one of the preceding claims, characterized in that the internal body is made in an irregular shape, and/or any other desired shape, particularly a ventricle-like or atrium-like or heart-like shape.

10. A mold according to any one of the preceding claims, characterized in that the external body comprises at least one opening in fluid communication with the cavity.

11. A mold according to claim 10, characterized in that the external body comprises a first opening in fluid communication with the cavity for receiving there through a first conduit for guiding fluid to the cavity, and a second opening in fluid communication with the cavity for receiving there through a second conduit for guiding fluid away from the cavity.

12. A mold according to claim 11, characterized in that the external body comprises an inlet wall in which inlet wall both the first and second opening are provided.

13. Method for preparing a hollow 3D cell tissue structure such as an organoid, in particular a human heart mimic, comprising the steps, particularly consecutive steps, of:
preparing an external body having an inner surface delimiting a cavity in the external body;
preparing an internal body of fugitive material in a predefined shape and having an outer surface;
placing the internal body inside the cavity of the external body, wherein the inner surface and outer surface define a culture chamber there between;
providing cells, particularly stem cells, progenitor cells, cardiomyocytes and/or other cardiac cells into the culture chamber;
subjecting the cells to incubating conditions for a period of time to promote the formation of a supported cell structure, and/or to incubating conditions to promote the formation of a cell tissue, in the culture chamber around the outer surface of the internal body;
subjecting the internal body of fugitive material to a condition whereto the fugitive material reacts thereby degrading the internal body for removal from the cavity without significantly affecting an integrity of a formed supported cell structure or cell tissue in the culture chamber and maintaining a functioning of the supported cell structure or cell tissue.

14. Method according to claim 13, wherein the step of subjecting the internal body to a condition whereto the fugitive material reacts comprises liquefying and/or dissolving of the internal body by a change in temperature, particularly comprises dissolving the internal body by increasing the temperature from a start temperature below 10 degrees Celsius to a degradation temperature between 10 and 40 degrees Celsius.

15. Method according to claim 13 or claim 14, wherein the internal body is liquefied and/or dissolved by supplying a liquefying or dissolving agent to the internal body.

16. Method according to claim 15, wherein said liquefying or dissolving agent comprises an enzyme or chemical compound or composition.

17. Method according to claim 13 or claim 14, wherein the internal body is liquefied and/or dissolved by sonication and/or in the presence of light with a wavelength above or below a threshold value.

18. Method according to any one of claims 13-17, wherein the step of providing cells in the culture chamber comprises providing cells in the cavity of the external body followed by placing of the internal body inside the cavity of the external body to position the cells in the culture chamber.

19. Method according to any one of claims 13-18, wherein the preparing of the external body comprises preparing a first part of the external body comprising a first section of the cavity, preparing a separate second part of the external body comprising a second section of the cavity, and coupling of the first part and second part to each other such that the first section of the cavity and the second section of the cavity communicate with each other thereby forming the cavity.

20. Use of a mold according to any one of claims 1-12 for preparing a hollow 3D cell tissue structure, preferably an organoid with a cavity.

21. Use according to claim 20, wherein said organoid is a cardiac organoid, preferably a human cardiac organoid.

22. A bioreactor comprising:
a mold according to any one of claims 1-12; and
cells that are present in the culture chamber between the inner surface of the external body and the outer surface of the internal body of said mold.

23. A hollow 3D cell tissue structure obtainable by a method according to any one of claims 13-19.
